(19) United States
(12) Patent Application Publication
(10) Pub. No.: US 2012/0290058 A1
(43) Pub. Date: Nov. 15, 2012

(54) METHODS AND SYSTEMS FOR THE TREATMENT OF ANXIETY DISORDERS AND DISORDERS WITH PSYCHOTIC FEATURES

(75) Inventors: Jean-Philippe Langevin, Tucson, AZ (US); Scott E. Krahl, Stevenson Ranch, CA (US); Antonio Alfonso Ferreira Desalles, Los Angeles, CA (US)

(73) Assignees: United States Government Department of Veterans Affairs, Washington, DC (US); The Regents of the Univeristy of California, Oakland, CA (US)

(21) Appl. No.: 13/513,436

(22) PCT Filed: Dec. 2, 2010
(86) PCT No.: PCT/US10/58780

§ 371 Date: Aug. 1, 2012

Related U.S. Application Data
(60) Provisional application No. 61/266,057, filed on Dec. 2, 2009.

(51) Int. Cl. A61N 1/05

(52) U.S. Cl. 607/116

(57) ABSTRACT

Methods and systems for treating anxiety disorders and disorders with psychotic features include implanting at least one electrode in the patient so as to contact a predetermined region of the brain, coupling the at least one electrode to a signal generating source and transmitting an electrical signal from the source to the predetermined region of the brain through the at least one electrode. In one aspect, the predetermined region of the brain is the amygdala, and more specifically, the basolateral nucleus.

### CROSS REFERENCE TO RELATED APPLICATIONS

This application claims the benefit under 35 USC §119(e) to U.S. Provisional Patent Application No. 61/266,057, filed Dec. 2, 2009 and titled “Methods and Systems for the Treatment of Anxiety Disorders”, which is hereby incorporated by reference in its entirety.

### BACKGROUND

The present disclosure relates to methods and systems used for the treatment of mood and/or anxiety disorders and disorders with psychotic features via deep brain stimulation. More specifically, methods and systems for the treatment of post-traumatic stress disorder by deep brain stimulation are provided.

Anxiety disorder is a family of disease characterized by abnormal (pathologic) fear and/or anxiety. The family of anxiety disorders includes generalized anxiety disorders, panic disorder, post-traumatic disorder (“PTSD”), phobias, and obsessive-compulsive disorder (“OCD”). Among these, PTSD is an anxiety disorder that occurs following the experience or witnessing of a life-threatening or integrity-threatening event. It is characterized by the presence of three components: re-experiencing, avoidance and arousal. Re-experiencing refers to the vivid sense of reliving the traumatic event emotionally (fear and panic), cognitively (imagery) and physiologically (tachycardia, sweating). Re-experiencing leads to avoidance. Nevertheless, as re-experiencing occurs rather unexpectedly, avoidance generalizes and the subject will avoid stimuli related to the trauma and stimuli not related to the trauma. Finally, arousal describes a constellation of symptoms such as insomnia and difficulty concentrating.

These symptoms are greatly disabling and may lead to increased risk for unemployment and suicide. The lifetime prevalence of PTSD is estimated to be as high as about 1 in 12 people. This makes PTSD the fourth most prevalent mental illness in young adults after depression, drug dependence and phobia. Moreover, in the US, the problem is particularly important given the large population of war veterans. A recent study from the Veterans Administration (VA) revealed a lifetime prevalence of full PTSD of 30.9% among male veterans and 26% for female veterans, and the prevalence of partial PTSD revealed was 22.5% and 21.2% for male and female veterans, respectively.

The current therapy for PTSD includes a cognitive-behavioral arm consisting of exposure therapy and relaxation techniques, as well as a pharmacological arm consisting mainly of antidepressant medication such as paroxetine (Paxil®). Despite these options, some studies report that over 33% of patients will remain non-responsive to therapy.

Disorders with psychotic features, such as paranoia and psychosis, may include schizophrenia, psychotic episodes of depression and bipolar disorder, and cocaine or other stimulant-induced psychosis. Current treatment of these disorders may be by anti-psychotic medications. These medications are usually very effective, however, several limitations exist, including: certain patients are unresponsive to the medications, the therapy requires daily compliance with medication intake, and there are long-term and potentially disabling side effects (e.g. tardive dyskinesia) associated with long-term use of these medications.

Therefore, a need exists for alternative methods and systems for treating PTSD and other anxiety disorders and disorders with psychotic features.

### SUMMARY

One aspect of the subject matter of the present disclosure addresses the aforementioned needs by providing a method of treating PTSD by deep brain stimulation of neural structures postulated to mediate various brain functions.

In another aspect of the present disclosure, there is provided a method of treating PTSD by stimulation of a specific neural structure, the amygdala.

In another aspect of the present disclosure, there is provided a method of treating a patient exhibiting PTSD symptoms by stimulating a predetermined area of a target amygdala that exhibits altered activity relative to its counterpart.

In another aspect of the present disclosure, there is provided a method of treating an anxiety disorder or a disorder with psychotic features in a patient. In various embodiments, the method includes: implanting an electrode assembly in the patient so as to contact a predetermined region of the brain, coupling the electrode assembly to a signal generating source; and transmitting an electrical signal from the source to the predetermined region of the brain through the electrode assembly to treat an anxiety disorder or a disorder with psychotic features in the patient. In some embodiments, the predetermined region is a region of an amygdala. In some embodiments, the region of the amygdala is the basolateral nucleus, the nuclei of a cortical complex or a centromedial complex. In some embodiments, the anxiety disorder is post-traumatic stress disorder, panic disorders, phobias or obsessive-compulsive disorder. In some embodiments, the disorder with psychotic features is selected from the group consisting of: schizophrenia, psychotic episodes of depression and bipolar disorder, and cocaine or other stimulant-induced psychosis. In some embodiments, the electrode assembly comprises at least one electrode. In some embodiments, the electrode assembly comprises a first electrode configured to stimulate a first region of a patient's brain and a second electrode configured to stimulate a second region of a patient's brain. In some embodiments, the first region is a basolateral nucleus of a right amygdala and the second region is a basolateral nucleus of a left amygdala. In some embodiments, transmitting an electrical signal comprises stimulating the basolateral nucleus of the target amygdala at the following parameters: a stimulus frequency between about 90 Hz and about 300 Hz, with a pulse duration between 20 μsec and 300 μsec for a continuous period of time and at a voltage range between about IV and about 7V and/or amperage of about 0.1 mA to 30 mA. In some embodiments, transmitting an electrical signal comprises stimulating the basolateral nucleus of the target amygdala at the following parameters: a stimulus frequency between about 150 Hz and about 180 Hz, with a pulse duration between 120 μsec and 180 μsec for a continuous period of time. In some embodiments, transmitting an electrical signal comprises stimulating the basolateral nucleus of the target amygdala at the following parameters: a stimulus frequency of about 160 Hz with a pulse duration of about 120 μsec and an amperage of about 1.5 mA and/or a voltage of 2.5 V.

In another aspect of the present disclosure, there is provided a method of treating an anxiety disorder or a disorder with psychotic features in a patient. In various embodiments, the method includes stimulating a predetermined region of the patient's brain to modulate the activity of the predetermined region. In some embodiments, the stimulating step is performed by an implanted electrode assembly coupled to a signal generating source, the assembly in contact with or in close proximity to the predetermined region, wherein the predetermined region is an amygdala and wherein the electrode assembly comprises at least one electrode configured to stimulate a basolateral nucleus in a right amygdala and at least one electrode configured to modulate a basolateral nucleus in a left amygdala. In some embodiments, the anxiety disorder is post-traumatic stress disorder.

In another aspect of the present disclosure, an implantable stimulation system configured to stimulate the basolateral nucleus of a target amygdala of a patient to treat an anxiety disorder or a disorder with psychotic features is disclosed. In various embodiments, the system includes a pulse or signal generating source and an electrode assembly coupled to the source by an electrically conductive connecting element, wherein the assembly comprises one or more electrodes configured to stimulate the basolateral nucleus of the target amygdala to treat an anxiety disorder or a disorder with psychotic features. In some embodiments, the system further comprises a transcutaneous programming device configured for placement on the skin overlying the signal generating source, the device configured to control the signal generating source. In some embodiments, the anxiety disorder is post-traumatic stress disorder. In some embodiments, the electrode assembly is configured to stimulate both the left and right amygdala.

In another aspect of the present disclosure, there is provided a method of treating a patient exhibiting post-traumatic stress disorder symptoms. The method includes: exposing the patient to a simulated post-traumatic stress disorder triggering event, determining the activity of the amygdalae of the brain, implanting at least two electrodes in the patient so as to contact a predetermined site of each of a right amygdala and a left amygdala, and stimulating the predetermined site of each amygdala to modulate its activity. In one aspect, the predetermined site of each of the right amygdala and the left amygdala is the basolateral nucleus.

### BRIEF DESCRIPTION OF THE DRAWINGS

The present disclosure, both as to its organization and manner of operation, may be understood by reference to the following description, taken in connection with the accompanying drawings, in which:

FIG. 1A is a cross-sectional side view of the brain, showing the major neuronal structures and the relative location of the amygdalae;

FIG. 1B is a cross-sectional view of the amygdala, showing various amygdaloid nuclei;

FIG. 2 shows a schematic view of an implantable stimulation system configured to stimulate a predetermined site of a target amygdala in a method of treating PTSD provided according to aspects of the present disclosure;

FIG. 3 shows a three-dimension model of the planned trajectories of two intracranial electrodes configured to stimulate a predetermined site of a target amygdala according to various embodiments of the present disclosure;

FIG. 4 depicts the electrode trajectories of FIG. 3 superimposed on the brain MRI of the patient in a probe view;

FIG. 5 depicts the electrode trajectories of FIG. 3 superimposed on the brain MRI of the patient in a sagittal view; and

FIG. 6 is a flowchart illustrating a method of treating a patient exhibiting PTSD symptoms by stimulating a predetermined site of a target amygdala according to aspects of the present disclosure.

### DETAILED DESCRIPTION

The following description is provided to enable any person skilled in the art to make and use the subject matter of this disclosure, and it sets forth the best modes contemplated for carrying out the various aspects of the disclosure. Various modifications, however, will remain readily apparent to those skilled in the art, since the generic principles of the disclosed subject matter have been defined herein specifically to describe: (1) a method of treating PTSD and other anxiety disorders and disorders with psychotic features by deep brain stimulation of specific neural structures; (2) a method of treating PTSD and other anxiety disorders and disorders with psychotic features by stimulation of a specific neural structure, the amygdala; and (3) a method of treating a patient exhibiting PTSD symptoms, or symptoms of other anxiety disorders and disorders with psychotic features, by stimulating a predetermined area of a target amygdala that exhibits altered activity relative to its counterpart.

The pathogenesis of PTSD is not clearly understood. Approximately 25% of people exposed to a traumatic event will develop PTSD symptoms and only 25% of those will go on to demonstrate chronic PTSD. These values suggest an interaction between an underlying predisposition in certain individuals and a traumatic event.

Neuroimaging studies have shed some light on the pathogenesis of PTSD, wherein a higher activity of the amygdalae has been shown in PTSD patients compared to a control group. It has been postulated that PTSD may be caused by hyperactivity of at least one of the amygdalae.

Without wishing to be bound by any particular theory, over-activation of the amygdala may also play a role in other pathological conditions, including psychiatric conditions with symptoms of paranoia and psychosis. It has been postulated that the neurotransmitter dopamine is involved in driving amygdala activity. More specifically, it is thought that dopamine reduces inhibitory activity and augments excitatory activity leading to an overall increase in amygdala activity. Dopamine's action on the amygdala may at least partially mediate the symptoms of paranoia and psychosis described in psychiatric conditions, such as schizophrenia, psychotic episodes of depression and bipolar disorder, and cocaine or other stimulant-induced psychosis. While the remainder of this disclosure is generally discussed in terms of methods of treating PTSD, it is understood that the methods and systems disclosed herein may also be used to treat other anxiety disorders and disorders with psychotic features as disclosed herein. That is, the systems and methods disclosed herein may be used to modulate the activity of a specific region of the brain, such as the amygdala, thereby reducing the symptoms of PTSD, other anxiety disorders and disorders having psychotic features.

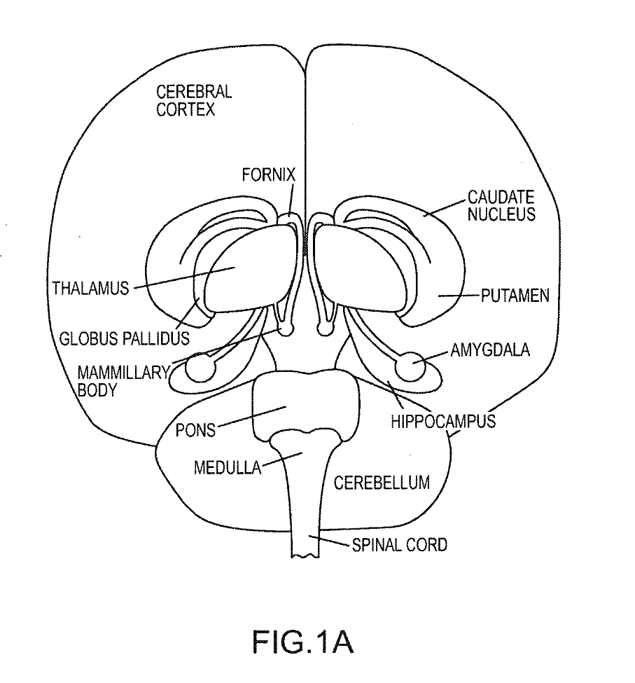

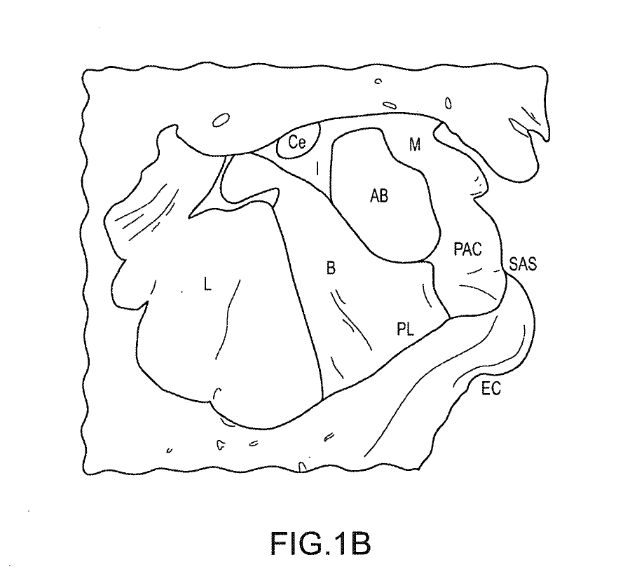

FIG. 1A, showing a cross-sectional side view of the brain, is reproduced from http://en.wikipedia.org/wiki/Amygdala. As shown in FIG. 1A, the amygdala is a complex mass of gray matter buried in the anterior-medial portion of the temporal lobe, anterior to the hippocampus. FIG. 1B, showing a coronal section of an amygdala, is reproduced from: Freese J, Amaral D., Neuroanatomy of the primate amygdala in The Human Amygdala, ed. Whalen P and Phelps E. The Guilford Press, 2009, p. 34. As shown in FIG. 1B, the amygdala comprises several nuclei. The lateral nucleus (complex L in FIG. 1B) mainly receives inputs from the higher-level sensory cortices. This information is then transmitted to the basal nucleus (complex B in FIG. 1B). The basal nucleus acts as a relay nucleus within the amygdala, receiving inputs from the lateral nucleus and sending outputs to the central nucleus (complex Ce in FIG. 1B). The basal nucleus also closes the loop between the amygdala and the sensory cortices. It receives sensory inputs for the lateral nucleus, and, in turn, it sends outputs back to the higher sensory cortices as well as the medial prefrontal cortex. The role of the basal nucleus as a relay nucleus makes it a prime target for neuromodulation. Finally the main output from the amygdala comes from the central nucleus which sends projections to the brainstem and the hypothalamus in order to translate the amygdalary activity into physiological conditions. The basolateral nucleus refers to the complex made of the lateral nucleus and the basal nucleus (L and B in FIG. 1B).

According to one aspect of the present disclosure, a method of treating PTSD by stimulation of at least one area of the brain is provided. Broadly speaking, the method of treating PTSD by deep brain stimulation comprises stimulating at least one specific region of the brain (the “target region”) to modulate the activity of the target region. In various embodiments, the method of treating PTSD by deep brain stimulation comprises implanting a stimulation device (e.g. an electrode assembly) in contact with, or within a predefined distance from, the target region, so that it is positioned to communicate a stimulation signal (e.g. by a neurostimulator in electrical communication with an electrode/electrode assembly) to the target region of the brain; and transmitting electrical signals to the target region through the stimulation device to stimulate the target region of the brain. In various embodiments, the stimulation to the target region is performed transcutaneously wherein electrical signals are generated from a stimulation device positioned outside the patient's body. In alternative embodiments, the stimulation of the target region of the brain is achieved non-invasively, such as by ultrasonic stimulation or transcranial magnetic stimulation. In various embodiments, the method of treating PTSD comprises implanting the stimulation device such that it is positioned to communicate a stimulation signal to an amygdala of the brain, preferably to a predetermined region or site of a target amygdala. In an alternative embodiment, the method of treating PTSD comprises implanting two stimulation devices, one positioned to communicate a stimulation signal to a predetermined region of the amygdala, and the other positioned to communicate a stimulation signal to the medial prefrontal cortex. In still another embodiment, the method of treating PTSD includes implanting two stimulation devices into the brain, one positioned to communicate with the right amygdala and one positioned to communicate with the left amygdala. In various embodiments, the neurostimulation of the amygdala is used as an adjunct to psychotherapy and/or pharmacotherapy for the treatment of PTSD. In this embodiment, amygdala neurostimulation facilitates the participation in psychotherapy and/or pharmacotherapy. In another embodiment, a recording electrode is receiving input from predetermined brain regions and transmitting this signal to turn on the stimulating device to deliver electricity in the predetermined site in the brain.

The human amygdala measures on the average approximately 1.5-2.0 cm in diameter, which is much larger than the diameter of a typical electrode (about 1.5 mm). To optimize the desired effect, the stimulation is localized at amygdaloid complexes postulated to be involved in the pathophysiology of PTSD and other disorders as disclosed herein. Thus, in various embodiments, the stimulation signal can be directed to the basolateral nucleus of the target amygdala. As discussed above, the lateral nucleus is the main sensory input to the amygdala, and the basal nucleus is the main relay nucleus and connection to the medial prefrontal cortex. Taken together, the role of the basolateral nucleus is likely to attach emotional value (i.e. fear) to a stimulus. This information is then transmitted to the central nucleus to produce a physiological effect. Presumably, in PTSD, the amygdala, and specifically the basolateral nucleus, is overactive, and this leads to fear being abnormally associated to multiple innocuous stimuli, therefore leading to avoidance of all stimuli. Targeting the basolateral nucleus will directly affect the pathology of PTSD. It is postulated that the amygdala is overactive in other anxiety disorders and disorders with psychotic features as detailed herein, and accordingly, targeting the amygdala will directly affect the pathology of other anxiety disorders and disorders with psychotic features. In various embodiments, the basolateral nucleus is targeted for treatment of other anxiety disorders and disorders with psychotic features. In various embodiments, the central nucleus is targeted for treatment of other anxiety disorders and disorders with psychotic features. In various embodiments, the cortical nuclei is targeted for treatment of other anxiety disorders and disorders with psychotic features. In various embodiments, the lateral nucleus is targeted for treatment of other anxiety disorders and disorders with psychotic features. In various embodiments, the basal nucleus is targeted for treatment of other anxiety disorders and disorders with psychotic features.

Thus, to perform the deep brain stimulation of the target amygdala, according to one aspect of the present disclosure, the method of treating PTSD comprises positioning the implantable stimulation device (e.g. an electrode or electrode assembly) so as to be “in communication” with a predetermined region (site) of the target amygdala, and stimulating the predetermined region of the target amygdala with electrical signals at specified operational parameters (e.g. via a neurostimulator). In various embodiments, including the illustrated embodiment, the predetermined site of the target amygdala is the basolateral nucleus. In alternative embodiments, the predetermined site of the target amygdala includes nuclei of the cortical complex (the cortical complex is not shown in FIG. 1B) or the centromedial complex (complex formed from the central nucleus and the medial nucleus, respectively represented by Ce and M in FIG. 1B). In some embodiments, the predetermined site of the target amygdala may be the accessory basal nucleus (represented by AB in FIG. 1B) or the interstitial/internuclei region (represented by I in FIG. 1B) of the amygdala. In some embodiments, the predetermined site of the target amygdala may be another subregion of the amygdala showing hyperactivity or hypoactivity (e.g. as shown by functional neuroimaging or neuronal recording or other appropriate method).

Current studies suggest that stimulation of certain target regions or sites in the brain may worsen PTSD. For example, stimulation of the stria terminalis (an output from the amygdala) may cause depression and anxiety in a patient. Thus, in some embodiments, the target region is not the stria terminalis. In some embodiments, the activity of the basomedial frontal cortex (i.e. medial prefrontal cortex) is not blocked since this area modulates the activity of the amygdala.

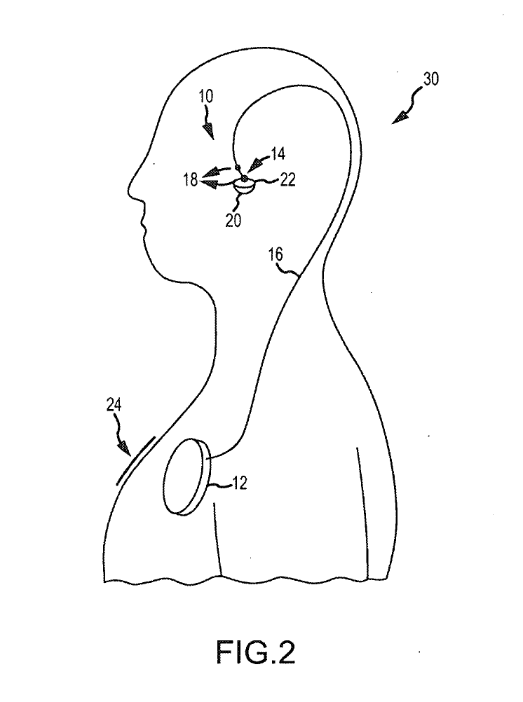

FIG. 2 shows a schematic view of an implantable stimulation system 10 configured to stimulate the basolateral nucleus of a target amygdala 20 of a patient 30 according to aspects of the present disclosure. The stimulation system 10 comprises a pulse or signal generating source 12 connected to a stimulation device, such as an electrode assembly 14, via an electrically conductive connecting element, such as a wire or cable 16. The electrode assembly 14 may comprise one or more electrodes 18 which interface with the basolateral nucleus of the target amygdala 20 via contact surfaces 22. In various embodiments, the signal generating source 12 is controllable by a transcutaneous programming device 24 placed on the skin overlying the signal generating source 12. In alternative embodiments, the signal generating source 12 may be controllable by a remote programming device. In some embodiments, the target amygdala may be both the left amygdala and the right amygdala. That is, in some embodiments, the implantable stimulation system 10 is configured to stimulate the basolateral nucleus of both the left and right amygdala (i.e. bilateral stimulation) according to aspects of the present disclosure. In such embodiments, the electrode assembly 14 may comprise one or more electrodes 18 which interface with the basolateral nucleus of each of the right amygdala and the left amygdala via contact surfaces 22. That is, and with reference to FIG. 1A, one or more electrodes 18 will interface with the basolateral nucleus of the right amygdala via contact surfaces 22 and one or more electrodes 18 will interface with the basolateral nucleus of the left amygdala via contact surfaces 22. The electrode assembly 14 is connected to the signal generating source 12 via an electrically conductive connecting element, such as a wire or cable 16. In some embodiments, the stimulating device is turned on by signals (a signal) recorded from specific areas of the brain when stimulation of the amygdalae becomes demanded by a patient's mental status of anxiety, panic, paranoia or other psychotic state.

In some embodiments, including the illustrated embodiments, the signal generating source 12 is implantable in the body of the patient 30. Any commercially available or modified implantable pulse generators may be used as the signal generating source 12, including, but not limited to, the Genesis® System from Advanced Neuromodulation System, Inc., model numbers 3604, 3608, 3609 and 3644. In various embodiments, the signal generating source 12 is implanted in a subcutaneous pocket located over the chest cavity (as shown in the illustrated embodiment), the abdomen or near the buttocks of the patient 30. In some embodiments, the signal generating source 12 may be implanted below the skin of a patient. In some embodiments, the stimulation source 12 may also be implanted inside a skull of the patient 30. In alternative embodiments, the signal generating source 12 may be located external of the patient's body. In various embodiments, the electrode assembly 14 may be configured to receive wireless signals from a wireless signal generator (not shown) external to the body of the patient 30. Examples of wireless signal generators include, but are not limited to, the Renew® System manufactured by Advanced Neuromodulation Systems, Inc., model numbers 3508 and 3516.

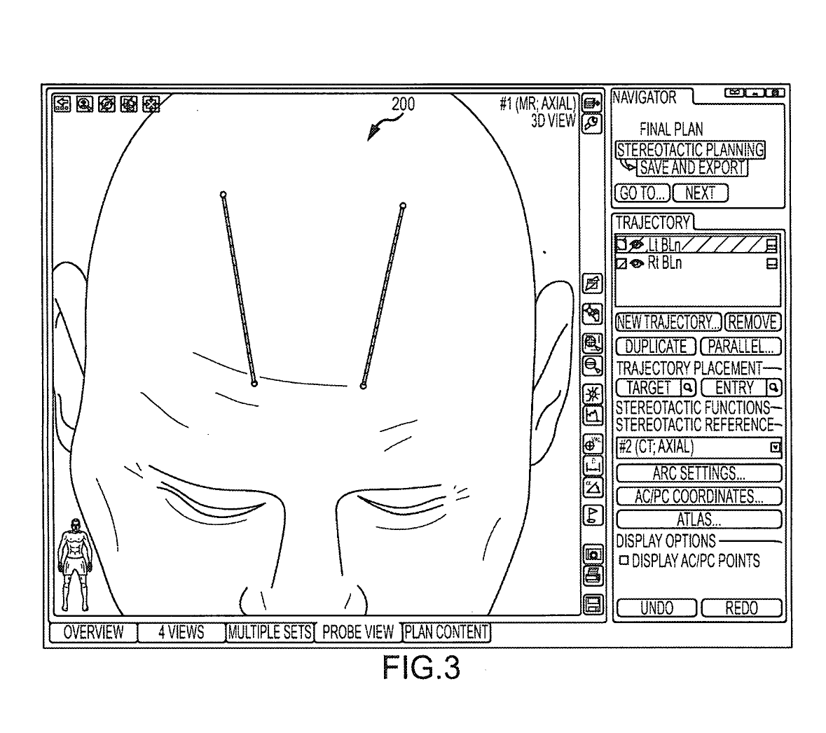

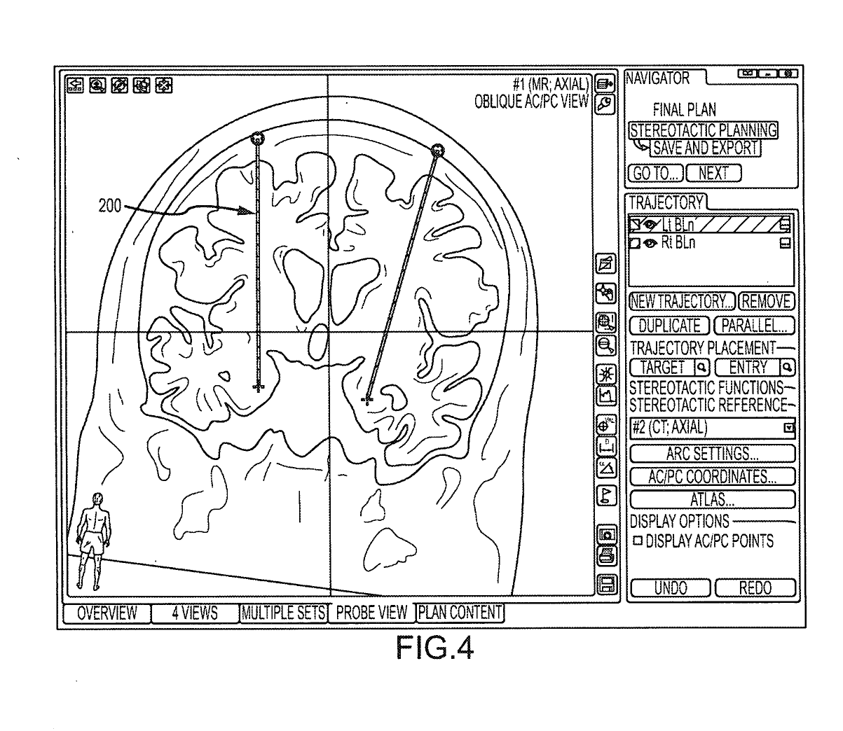

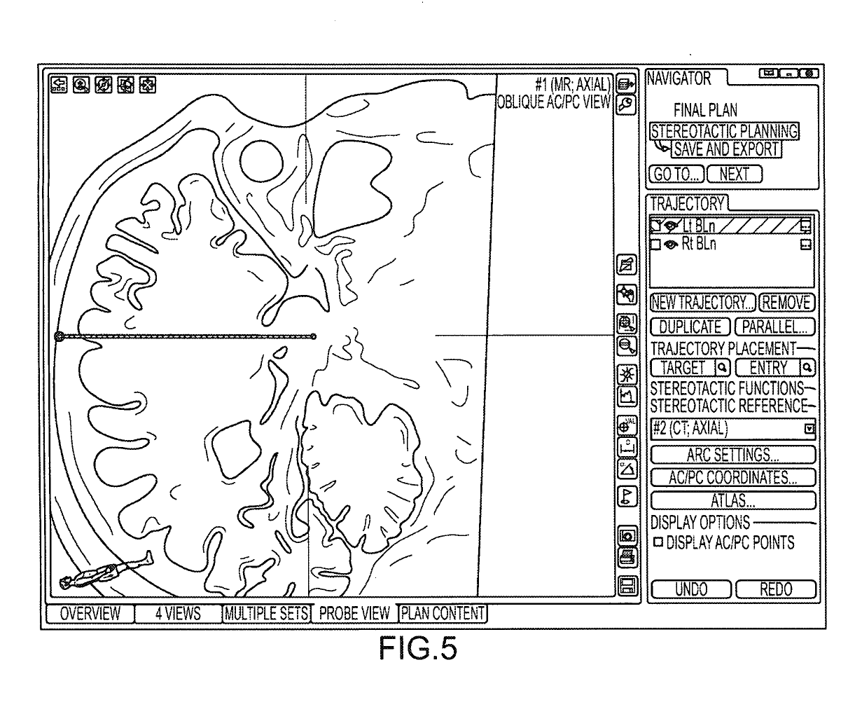

The electrode assembly 14 is placed in contact with the basolateral nucleus of the target amygdala by a surgical procedure. Placing electrodes in precise locations deep in the human brain is typically performed by stereotactic neurosurgery. Procedures that may be used for the precise placement of the electrode assembly 14 include, but are not limited to, targeting software and stereotactic head frames used in stereotactic neurosurgery. In various embodiments, and as depicted in FIGS. 3-5, the trajectories of the electrodes may be planned with targeting software from BrainLab (Feldkirchen, Germany). As shown in FIG. 3, which is a three-dimensional reconstruction of the trajectories of the electrode assembly, the entry points of the two intracranial electrodes are slightly anterior to the coronal suture. FIG. 4 illustrates a probe view of the electrode trajectories superimposed on the brain MRI of a patient. That is, the plane of the MRI is along the electrode trajectories. The orientation of the MRI is represented by the small man in the lower left corner. As can be understood from FIG. 4, the trajectory 200 is for the left electrode. Along this trajectory, the electrode avoids the ventricles and all major vessels, thus reducing the risk of intra-operative hemorrhage. FIG. 5 illustrates a sagittal probe view of the trajectories wherein the electrode trajectories are superimposed on the brain MRI of the patient. The orientation of the MRI is represented by the small man in the lower left corner. It can be appreciated that in other embodiment, other trajectories may be used.

After the electrode assembly 14 has been placed in contact with the basolateral nucleus of the target amygdala, it can be connected with the signal generating source 12 either via the connecting element 16 or wirelessly. A doctor, or the patient, or another person may input signal parameters to the electrode assembly 14 to stimulate the target site in the amygdala for the treatment of PTSD.

To stimulate the basolateral nucleus of the target amygdala, the signal generating source 12 is programmable to generate electrical signals at optimized operational parameters. In various embodiments, the signal generating source 12 is programmable to deliver high frequency signals to the basolateral nucleus of the target amygdala. Without being bound by any particular theory, based on clinical observations, it is postulated that low frequency stimulation activates a neural structure, whereas high frequency stimulation inhibits the function of the neural structure. The definition of “high frequency” is dependent on how the brain is stimulated. Generally, in the field of deep brain stimulation, the use of electrical signal frequencies higher than 100 Hz is considered high frequency stimulation. If transcranial magnetic stimulation is used, frequencies higher than 20 Hz are considered high frequency stimulation. In this case, as the hyperactivity of the amygdala is postulated to mediate PTSD, the basolateral nucleus of the amygdala is stimulated with high frequency electrical signals to inhibit its activity. In various embodiments, the basolateral nucleus of the target amygdala is stimulated at a stimulus frequency between about 100 Hz and about 250 Hz, with a pulse duration between 60 μsec and 210 μsec for a continuous period of time. The voltage of the stimulation is patient-specific and can range between about 1V and about 7V and/or an amplitude between 0.1 mA to 30 mA. In various embodiments, the neurostimulation of the basolateral nucleus is carried out at a stimulus frequency between about 150 Hz and about 180 Hz, with a pulse duration between 120 μsec and 180 μsec and a voltage of about 2.5 V and/or an amplitude of 1.5 mA for a continuous period of time. In various embodiments, the neurostimulation of the basolateral nucleus is carried out at a stimulus frequency of about 160 Hz with a pulse duration of about 120 μsec. Thus, in various embodiments, the method of treating PTSD according to aspects of the present disclosure comprises implanting a stimulation device such that the stimulation device is positioned to communicate with a predetermined site of a target amygdala at a location as disclosed herein, coupling the stimulation device with a pulse or signal generating source; and transmitting high frequency electrical signals from the signal generating source to the predetermined site via the stimulation device to stimulate the predetermined site of the target amygdala.

In various embodiments, the predetermined site of the target amygdala is its basolateral nucleus. In another embodiment, the predetermined site of the target amygdale is its basolateral complex. In still another embodiment, the predetermined site of the target amygdale may include the subnuclei of the basolateral complex. In alternative embodiments, the predetermined site of the target amygdale may include subnuclei of the cortical complex or the centromedial complex.

In various embodiments, the electrode assembly is placed in contact with the predetermined site of the target amygdala at the following coordinates: 20 mm lateral to AC/PC line, 20 mm inferior to AC/PC plane and 4 mm anterior to midcommisural point (AC is the anterior commissure; PC is the posterior commissure). In various embodiments, these coordinates are used to position the electrode in the approximate location of the BLn. In some embodiments, minor adjustments (e.g. between approximately 1-3 mm) are then made based on additional data including anatomical location, which may be determined from an MRI, and regional activation, which may be shown by functional neuroimaging. In other embodiments, other coordinates may be used.

In various embodiments, the predetermined site of the target amygdala is stimulated at a stimulus frequency between about 100 Hz and about 300 Hz, with a pulse duration between 90 μsec and 210 μsec for a continuous period of time, and a voltage range from approximately 1-7V and/or an amperage range from about 0.1 mA to about 30 mA. In some embodiments, the predetermined site of the target amygdala is stimulated at a stimulus frequency between about 100 Hz and about 300 Hz, with a pulse duration between 60 μsec and 210 μsec for a continuous period of time and a voltage range from approximately 1-7V and/or an amperage range from about 0.1 mA to about 30 mA. In some embodiments, such as those employed to induce neuronal learning, the neurostimulation is carried out for a period of time, for examples days or weeks, then discontinued for a period of time. In some embodiments, some subjects may need stimulation parameters that vary in frequency, amplitude and pulse width.

In various embodiments, the neurostimulation can be delivered at one or more frequencies, or within a range of frequencies. In various embodiments, the frequency may be delivered in a range with an upper limit of 300 Hz and a lower limit of 20 Hz. Nevertheless, there may be embodiments in which the neurostimulation is carried out in a range of frequencies greater than 300 Hz or in a range of frequencies less than 20 Hz.

In other embodiments, the neurostimulation may typically be set to be delivered at frequencies in any range within an upper limit of about 250 Hz and a lower limit of about 130 Hz. Nevertheless, there may be embodiments in which the neurostimulation is carried out in a range of frequencies greater than 250 Hz or in a range of frequencies less than 130 Hz.

In various embodiments, the neurostimulation is delivered at a specific pulse width or range of pulse widths. The stimulation may typically be set to deliver pulse widths in any range within a lower limit of about 30 μsec and an upper limit of about 210 μsec. Nevertheless, there may be embodiments in which the neurostimulation is carried out in a range of pulse widths greater than 210 μsec or in a range of pulse widths less than 60 μsec.

In various embodiments, the neurostimulation is delivered at a specific voltage or range of voltages. The stimulation may typically be set to deliver voltage in any range within a lower limit of about 1V and an upper limit of about 7V. Nevertheless, there may be embodiments in which the neurostimulation is carried out in a range of voltages greater than 7V or in a range of voltages less than 1V.

In various embodiments, the neurostimulation is delivered at a specific amperage or range of amperages. The stimulation may typically be set to deliver amperages in any range within a lower limit of about 0.1 mA and an upper limit of about 30 mA. Nevertheless, there may be embodiments in which the neurostimulation is carried out in a range of amperages greater than 30 mA or in a range of amperages less than 0.1 mA.

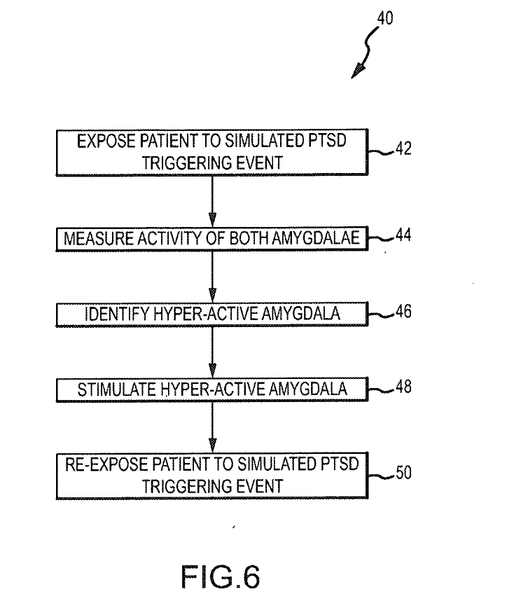

As set forth above, PTSD is an anxiety disorder that occurs following the experience or witnessing of a life-threatening or integrity-threatening event. Hyperactivity of at least one of the amygdalae has been postulated to mediate PTSD. Thus, in various embodiments, before the treatment is administered, the activity of the left and right amygdalae (FIG. 1A) is determined to identify the hyperactive or target amygdala. Furthermore, to evaluate the efficacy of the PTSD treatment the activity of the target amygdala needs to be monitored before and after treatment. In another embodiment, bilateral simulation, or stimulation of both the left and right amygdala is used for treatment. In some embodiments where bilateral stimulation is used, the activity of the amygdala is determined as described with respect to FIG. 6 and the method of treatment is also as described with respect to FIG. 6, except that electrodes are implanted in both amygdala and both amygdala may be stimulated. That is, in the case of bilateral stimulation, electrodes are implanted in both the left and right amygdala regardless of whether one or the other is the hyperactive amygdala. In such embodiments, the activity of the amygdalae may be determined to establish a baseline with which to provide a reference after implantation to evaluate the efficacy of the treatment. That is, the activity of the amygdalae may be determined or monitored pre-implantation, during implantation and post-implantation even though electrodes will be or have been implanted in both the left and right amygdala. During implantation, the activity of the amygdala may be measured by microelectrode recording or local field potential.

FIG. 6 is a flow chart illustrating a method 40 of treating a patient exhibiting PTSD symptoms by stimulating a predetermined site of a target amygdala according to aspects of the present disclosure. As shown in step 42, a patient suffering from PTSD is exposed to simulated conditions configured to trigger the patient's PTSD symptoms. The simulation can be accomplished by images, videos or behaviors, memories and/or cues associated with the traumatic event that initiated the anxiety disorder. At step 44, the activity of both the left and right amygdalae of the patient is measured. The activity of the amygdalae can be determined by techniques well-known to one of ordinary skill in the art, including, but not limited to, functional magnetic resonance imaging (fMRI) or positron emission tomography computerized tomography (PET-CT). From the analysis performed in step 44, a hyper-active or target amygdala is identified in step 46. In some embodiments, in the case where both amygdalae would be equally active, the right amygdala would be targeted. In embodiments where bilateral stimulation is used, both the left and right amygdalae are targeted, without regard to which amygdala is hyperactive. Treatment is initiated in step 48 by inserting one or more electrodes in a predetermined site of the target amygdala by procedures well known to one of ordinary skill in the art. In various embodiments where bilateral stimulation is used, treatment is initiated by inserting one or more electrodes in a predetermined site of both the right amygdala and the left amygdala. Step 48 also comprises coupling the one or more electrodes to a signal generating source. The stimulation in step 48 may include generating high frequency electrical signals (130-250 Hz) to the predetermined site of the target amygdala. In other embodiments, the stimulation in step 48 may include generating frequency electrical signals in a range between 20 Hz and 300 Hz to the predetermined site of the target amygdala. In various embodiments, the predetermined site of the target amygdala is stimulated at a stimulus frequency between about 130 Hz and about 250 Hz, with a pulse duration between 60 μsec and 210 μsec for a continuous period of time. In some embodiments, such as to induce neuronal learning, the neurostimulation is carried out for a period of time, for examples days or weeks, then discontinued for a period of time. In various embodiments, the neurostimulation of the predetermined site of the target amygdala is carried out at a stimulus frequency between about 150 Hz and about 180 Hz, with a pulse duration between 120 μsec and 180 μsec for a continuous period of time. In another embodiment, the neurostimulation is carried out at a stimulus frequency of about 160 Hz and with a pulse duration of about 120 μsec.

In various embodiments, the signal generating source is implantable within the patient's body. In some embodiments, the implantable signal generating source is programmable via a transcutaneous programming device placed on the body overlying the signal generating source. In some embodiments, the signal generating source may be external of the patient's body. In some embodiments, step 48 comprises stimulation of both the left and right amygdalae, such as by bilateral stimulation. In some embodiments, the stimulating device is turned on by signals (a signal) recorded from specific areas of the brain when stimulation of the amygdalae becomes demanded by a patient's mental status of anxiety, panic, paranoia or other psychotic state.

Following neurostimulation of the hyper-active amygdala, in step 50, to evaluate the efficacy of the treatment, the patient is re-exposed to the simulated conditions configured to trigger the patient's PTSD symptoms. The efficacy of the treatment may be determined by objectively and quantifiably comparing PTSD induced behavior of the patient before and after treatment. Objective and quantifiable data may include functional neuroimaging, ratings of PTSD behavior and self-assessment of quality of life. From the results obtained in step 50, operational parameters may be adjusted accordingly to optimize the treatment. The method 40 can then be repeated until the optimal operational parameters have been identified.

Thus, according to one aspect of the present disclosure, the method of treating a patient exhibiting PTSD symptoms by deep brain stimulation of a predetermined site of a target amygdala comprises exposing the patient to conditions configured to trigger the patient's PTSD symptoms, determining the activity of the amygdalae of the selected patient, identifying the hyper-active amygdala, and stimulating the predetermined site of the hyper-active or target amygdala with an implantable stimulation device at high frequency electrical signals. In various embodiments the predetermined site of the target amygdala is its basolateral nucleus. In another embodiment, the predetermined site of the target amygdale may include the subnuclei of the basolateral complex. In alternative embodiments, the predetermined site of the target amygdale may include subnuclei of the cortical complex or the centromedial complex. In alternative embodiments, the amygdala stimulation may be supplemented with medial prefrontal cortex stimulation.

Thus, according to another aspect of the present disclosure, a method of treating a patient exhibiting PTSD symptoms by deep brain stimulation of a predetermined site of a target amygdala comprises exposing the patient to conditions configured to trigger the patient's PTSD symptoms, determining the activity of the amygdalae of the selected patient, and stimulating the predetermined site of the target amygdala with an implantable stimulation device at high frequency electrical signals. In various embodiments the predetermined site of the target amygdala is its basolateral nucleus. In another embodiment, the predetermined site of the target amygdale may include the subnuclei of the basolateral complex. In alternative embodiments, the predetermined site of the target amygdala may include subnuclei of the cortical complex or the centromedial complex. In alternative embodiments, the amygdala stimulation may be supplemented with medial prefrontal cortex stimulation. In some embodiments, the target amygdala is both the left amygdalae and the right amygdalae. In another embodiment, the activity of the amygdala is measured with direct microelectrode recording or local field potential. With the microelectrode or the electrode in place, baseline recordings are obtained and then compared with recordings obtained with exposure to provocative stimuli (e.g. images, video, text, computer programs etc.). In this embodiment, the information obtained from such recording is then used to predict the post-operative parameters of stimulation. The information obtained can also be used to determine which amygdala to stimulate chronically or to preferentially stimulate.

The following example is presented to set forth more clearly the subject matter of this disclosure without imposing any limits on the scope thereof.

In one example, rats pre-conditioned to exhibit PTSD are treated by neurostimulation of the basolateral nucleus of a target amygdala provided according to aspects of the present disclosure.

The rat model of PTSD involving an inescapable shock paradigm has been previously established. In this model, a rat is subjected to a session of inescapable shocks (IS) representing a traumatic event. During the IS session, the rat is also exposed to an object serving as a cue. In a second time, the behavior of the rat is evaluated when it is re-exposed to the cue-object. Rats subjected to IS bury the object under the bedding of their cage since they are fearful of the object. The burying behavior is thought to be a sign of hyper-vigilance, an important sign of PTSD.

This study included two animal groups and ten total animals.
a. Deep brain stimulation (DBS) experimental group (five animals)
b. DBS-placebo control group (five animals)

Electrode placement: All the rats underwent the placement of a right amygdala electrode. The rats were anesthetized using an IV anesthetic and then placed in a rodent stereotactic frame. A linear incision was made along the midline of the skull down to the bone. A small burr hole was drilled lateral, to the right side and, using the stereotactic frame, the small electrode was inserted in the right basolateral nucleus of the amygdala through a right transfrontal approach. The electrode was fixed in place using a base made of three small screws and dental cement.

Seven days following the placement of the electrode, all the animals were subjected to the inescapable shock event. They were placed in a box with metallic floor. The shocks consisted of two 1-second trains of shock per minute for 5 minutes. Every shock train consisted of a 0.01 sec shock followed by 0.02 sec of rest. A total of 10 shocks were administered over the 5-minute period. Each shock was 2 mA.

Deep brain stimulation: All the rats were brought to the laboratory four hours everyday for seven days. All the rats had their intracranial electrode connected to an external pulse generator during those four hours. The control rats did not receive any current, whereas the DBS experimental rats were treated with high-frequency stimulation (160 Hz, 120 μsec, 2.5 volts). After seven days of treatment/placebo, the rats were tested for the object burying behavior.

Object-burying task: Following the seven days of stimulation (or control sham-stimulation), the rats were brought back to the laboratory. They were exposed, for 15 minutes, to the object which they encountered during the initial IS session. The behavior of the rats was observed and recorded during those 15 minutes. The time spent burying the object or playing/biting the object was recorded.

After the burying task, the rats were sacrificed. The brains of the rats in the DBS group were preserved in formalin and then cut and examined under the microscope to confirm the placement in the amygdala.

The results of the above-described experiments confirmed that deep brain stimulation of the target amygdala reduced the symptoms associated with PTSD in a rat model. The DBS-treated rats spent on average 12 times less time than control rats burying the object. The DBS-treated rats played with the object on average 18 times more time than the control rats.

Those skilled in the art will appreciate that various adaptations and modifications of the above-described embodiments of the disclosed methods and stimulation system are within the scope and spirit of the present disclosure. For example, deep brain stimulation of other nuclei of the amygdala for the treatment of PTSD is also within the scope of the present disclosure. Alternatively, simultaneous stimulation of a plurality of nuclei of the amygdala for the treatment of PTSD is also within the scope of the present disclosure. Also, combined stimulation of the medial prefrontal cortex and the amygdala is within the scope of the present disclosure. Also, stimulation of the target nucleus may be accomplished by application of energy in many forms, such as magnetic or ultrasonic. Also, adaptations and modifications of the above-described embodiments of the disclosed methods and systems for the treatment of anxiety disorders other than PTSD are within the scope and spirit of the present disclosure. Therefore, it is to be understood that the subject matter of this disclosure may be practiced other than as specifically described herein.

What is claimed is:

1. A method of treating an anxiety disorder or a disorder with psychotic features in a patient, comprising:
implanting an electrode assembly in the patient so as to contact a predetermined region of the brain;
coupling the electrode assembly to a signal generating source; and
transmitting an electrical signal from the source to the predetermined region of the brain through the electrode assembly to treat an anxiety disorder or a disorder with psychotic features in the patient.

2. The method of claim 1, wherein the predetermined region is a region of an amygdala.

3. The method of claim 2, wherein the region of the amygdala is selected from the group consisting of: basolateral nucleus, nuclei of a cortical complex and a centromedial complex.

4. The method of claim 2, wherein the region of the amygdala is the basolateral nucleus.

5. The method of claim 1, wherein the anxiety disorder is selected from the group consisting of post-traumatic stress disorder, panic disorders, phobias and obsessive-compulsive disorder.

6. The method of claim 1, wherein the anxiety disorder is post-traumatic stress disorder.

7. The method of claim 1, wherein the disorder with psychotic features is selected from the group consisting of: schizophrenia, psychotic episodes of depression and bipolar disorder, and cocaine or other stimulant-induced psychosis.

8. The method of claim 1, wherein the electrode assembly comprises at least one electrode.

9. The method of claim 1, wherein the electrode assembly comprises a first electrode configured to stimulate a first region of a patient's brain and a second electrode configured to stimulate a second region of a patient's brain.

10. The method of claim 9, wherein the first region is a basolateral nucleus of a right amygdala and the second region is a basolateral nucleus of a left amygdala.

11. The method of claim 4, wherein transmitting an electrical signal comprises stimulating the basolateral nucleus of the target amygdala at the following parameters: a stimulus frequency between about 100 Hz and about 300 Hz, with a pulse duration between 30 μsec and 210 μsec for a continuous period of time and at a voltage range between about 1V and about 7V and/or amplitude between about 0.1 mA and about 30 mA.

12. The method of claim 4, wherein transmitting an electrical signal comprises stimulating the basolateral nucleus of the target amygdala at the following parameters: a stimulus frequency between about 150 Hz and about 180 Hz, with a pulse duration between 120 μsec and 180 μsec for a continuous period of time.

13. The method of claim 4, wherein transmitting an electrical signal comprises stimulating the basolateral nucleus of the target amygdala at the following parameters: a stimulus frequency of about 160 Hz with a pulse duration of about 120 μsec.

14. A method of treating an anxiety disorder or a disorder with psychotic features in a patient comprising: stimulating a predetermined region of the patient's brain to modulate the activity of the predetermined region.

15. The method of claim 14, wherein the stimulating step is performed by an implanted electrode assembly coupled to a signal generating source, the assembly in contact with or in close proximity to the predetermined region, wherein the predetermined region is an amygdala and wherein the electrode assembly comprises at least one electrode configured to stimulate a basolateral nucleus in a right amygdala and at least one electrode configured to modulate a basolateral nucleus in a left amygdala.

16. The method of claim 15, wherein the anxiety disorder is post-traumatic stress disorder.

17. An implantable stimulation system configured to stimulate the basolateral nucleus of a target amygdala of a patient to treat an anxiety disorder or a disorder with psychotic features, the system comprising:
a pulse or signal generating source; and
an electrode assembly coupled to the source by an electrically conductive connecting element, wherein the assembly comprises one or more electrodes configured to stimulate the basolateral nucleus of the target amygdala to treat an anxiety disorder or a disorder with psychotic features.

18. The system of claim 17, further comprising a transcutaneous programming device configured for placement on the skin overlying the signal generating source, the device configured to control the signal generating source.

19. The system of claim 17, wherein the anxiety disorder is post-traumatic stress disorder.

20. The system of claim 17, wherein the electrode assembly is configured to stimulate both the left and right amygdala.
